(19) United States
(12) Patent Application Publication
(10) Pub. No.: US 2018/0160993 A9
(43) Pub. Date: Jun. 14, 2018

(54) X-RAY IMAGING APPARATUS AND METHOD FOR CONTROLLING THE SAME

(71) Applicant: SAMSUNG ELECTRONICS CO., LTD., Suwon-si (KR)

(72) Inventors: Jae Hak LEE, Yongin-si (KR); Dong-Goo KANG, Hwaseong-si (KR); Young Hun SUNG, Hwaseong-si (KR); Jae Mock YI, Hwaseong-si (KR)

(73) Assignee: SAMSUNG ELECTRONICS CO., LTD., Suwon-si (KR)

(21) Appl. No.: 14/868,673

(22) Filed: Sep. 29, 2015

(30) Foreign Application Priority Data
Jan. 20, 2015 (KR) 10-2015-0009234

(51) Int. Cl. A61B 6/00; G06K 9/62; G06K 9/46; G06T 5/00; G06T 5/50

(52) U.S. Cl. CPC A61B 6/52; A61B 6/461; G06K 9/6202; G06K 9/469; G06K 2009/6213; G06K 9/4647; G06T 5/002; G06T 5/50; G06T 2207/10116; G06K 9/4661

(57) ABSTRACT

An X-ray imaging apparatus includes an image comparator configured to compare a first frame image obtained by scanning an object at a particular point of time, and one or more second frame images obtained by scanning the object before the particular point of time, and an image restorer configured to restore a background region of the first frame image based on a result of the comparison between the first frame image and the one or more second frame images.

### CROSS-REFERENCE TO RELATED APPLICATION

This application claims priority from a Korean Patent Application No. 10-2015-0009237, filed on Jan. 20, 2015 in the Korean Intellectual Property Offices, the disclosure of which is incorporated herein in its entirety by reference.

### BACKGROUND

1. Field

Apparatuses and methods consistent with exemplary embodiments relate to an X-ray imaging apparatus, which irradiates X-rays to an object to generate X-ray images, and a method for controlling the same.

2. Description of the Related Art

X-ray imaging apparatuses are devices for obtaining an image of the inside of an object, such as a human body or a thing, by irradiating X-rays to the object and detecting X-rays that passed through the object. Since the transmittance of X-rays varies depending on characteristics of a material included in the object, an internal structure of the object may be imaged by detecting intensity or strength of the X-rays that penetrate the object. An internal structure of an object can be easily detected with the X-ray imaging apparatus, and thus, the X-ray imaging apparatus may be used, for example, to detect abnormalities, such as lesions, in a patient in medical fields, or to examine the inside of baggage in the airport.

### SUMMARY

One or more exemplary embodiments provide an X-ray imaging apparatus and a method for controlling the same in which a background region of a current frame image may be restored more precisely by using pixel information of pixels contained in a previously captured frame image.

In accordance with an aspect of an exemplary embodiment, provided is an X-ray imaging apparatus including an image comparator configured to compare a first frame image obtained by scanning an object at a particular point of time, and one or more second frame images obtained by scanning the object before the particular point of time; and an image restorer configured to restore a background region of the first frame image based on a result the comparison between the first frame image and the one or more second frame images.

The image comparator is configured to map, pixel by pixel, pixel information of the background region of the first frame image and pixel information of a region of interest of the one or more second frame images.

The image comparator is configured to generate a graph including one or more points, each of the one or more points being mapped, per pixel, between the pixel information of the background region of the first frame image and the pixel information of the region of interest of the one or more second frame images.

The image comparator is configured to determine at least one of a brightness level per pixel and a noise level per pixel of the background region of the first frame image, based on the graph.

The image comparator is configured to determine a constraint value for at least one of a brightness level per pixel and a noise level per pixel of the background region of the first frame image, based on the graph.

The image restorer is configured to restore the background region of the first frame image by using brightness levels of the background region of the first frame image that are determined based on the result of the comparison between the first frame image and the one or more second frame images.

The image restorer is configured to restore the background region of the first frame image by using noise levels of the background region of the first frame image that are determined based on the result of the comparison between the first frame image and the one or more second frame images.

The image restorer is configured to restore the background region of the first frame image by using a constraint value for at least one of a brightness level and a noise level of the background region of the first frame image, the constraint value being determined based on the result of the comparison between the first frame image and the one or more second frame images.

The X-ray imaging apparatus may further include a display configured to display the restored first frame image.

In accordance with an aspect of another exemplary embodiment, provided is an X-ray imaging apparatus including: an image storage configured to store pixel information of one or more second frame images obtained by scanning an object before a particular point of time; and an image generator configured to generate a first frame image obtained by scanning the object at the particular point of time, by using the pixel information of the stored one or more second frame images.

The image generator may be configured to generate the first frame image by using a result of summing, pixel by pixel, pixel information of the stored one or more second frame images.

The image generator may be configured to calculate, pixel by pixel, a sum of respective brightness levels of pixels of the one or more second frame images, and generate the first frame image by using the sum of the respective brightness levels of the pixels of the one or more second frame images.

The image generator may be configured to calculate, pixel by pixel, a sum of respective noise levels of pixels of the one or more second frame images, and generate the first frame image by using the sum of the respective noise levels of the pixels of the one or more second frame image.

The X-ray imaging apparatus may further include a display configured to display the generated first frame image.

In accordance with an aspect of still another exemplary embodiment, provided is a method for controlling an X-ray imaging apparatus, the method including: comparing a first frame image obtained by scanning an object at a particular point of time, and one or more second frame images obtained by scanning the object before the particular point of time; and restoring a background region of the first frame image based on a result of the comparing.

The comparing may include mapping, pixel by pixel, pixel information of a background region of the first frame image and pixel information of a region of interest of the one or more second frame images.

The comparing may include generating a graph including one or more points, each of the one or more points being mapped, per pixel, between the pixel information of the background region of the first frame image and the pixel information of the region of interest of the one or more second frame images.

The comparing may include determining at least one of a brightness level per pixel and a noise level per pixel of the background region of the first frame image, based on the graph.

The comparing may include determining a constraint value for at least one of a brightness level per pixel and a noise level per pixel of the background region of the first frame image, based on the graph.

The restoring may include restoring the background region of the first frame image by using brightness levels of the background region of the first frame image determined based on the result of the comparing.

The restoring may include restoring the background region of the first frame image by using noise levels of the background region of the first frame image determined based on the result of the comparing.

The restoring may include restoring the background region of the first frame image by using a constraint value for at least one of a brightness level and a noise level of the background region of the first frame image, the constraint value being determined based on the result of the comparing.

In accordance with an aspect of still another exemplary embodiment, provided is a method for controlling an X-ray imaging apparatus, the method including: storing pixel information of one or more second frame images obtained by scanning an object before a particular point of time; and generating a first frame image obtained by scanning the object at the particular point of time, by using the pixel information of the stored one or more second frame images.

The generating may include generating the first frame image by using a result of summing, pixel by pixel, pixel information of the stored one or more second frame images.

The generating may include calculating, pixel by pixel, a sum of respective brightness levels of pixels of the one or more second frame images, and generating the first frame image by using the calculated sum of the brightness levels of the pixels of the one or more second frame images.

The generating may include calculating, pixel by pixel, a sum of respective noise levels of pixels of the one or more second frame images, and generating the first frame image by using the calculated sum of the noise levels of the pixels of the one or more second frame images.

In accordance with an aspect of still another exemplary embodiment, provide is an X-ray imaging apparatus including: an image acquirer configured to acquire a plurality of frame images of an object at different times; and an image processor configured to map a pixel in a background region of a current frame image to a pixel in a region of interest of at least one previous frame image, and compensate the background region of the current frame image based on pixel information of a mapping pixel in the region of interest of the at least one previous frame image.

The image processor may be configured to compensate the background region of the current frame image based on at least one of a difference between brightness levels between noise levels of mapping pixels between the current frame image and the at least one previous frame image and a difference between noise levels of mapping pixels between the current frame image and the at least one previous frame image.

The image processor may be configured to map pixels at the same coordinates between the background region of the current frame image and the region of interest of the at least one previous frame image.

When the current frame image and the at least one previous frame image correspond to different regions of the object, the image processor may be configured to perform an image coordination process to adjust coordinates of pixels of the current frame image and the at least one previous frame image, and the image processor is configured to map pixels at the same coordinates between the current frame image and the at least one previous frame image on which the image coordination process is performed.

### BRIEF DESCRIPTION OF THE DRAWINGS

The above and/or other aspects will become more apparent by describing certain exemplary embodiments with reference to the accompanying drawings in which:

FIG. 1 illustrates an exterior appearance of an X-ray imaging apparatus according to an exemplary embodiment;

FIG. 2 illustrates an exterior appearance of an X-ray imaging apparatus including a console device, a display device, and an input device, according to an exemplary embodiment;

FIG. 3 illustrates a cross-section of an X-ray generator according to an exemplary embodiment;

FIG. 4 is a block diagram of an X-ray imaging apparatus according to an exemplary embodiment;

FIGS. 5A and 5B illustrate frame images before and after restoration of a background region according to an exemplary embodiment;

FIG. 6 illustrates a region of interest that moves in time, according to an exemplary embodiment;

FIGS. 7A and 7B illustrate a relationship between a first frame image and a second frame image that are captured at different points of time, according to an exemplary embodiment;

FIG. 8 illustrates an example of mapping pixel information between a first frame image and a second frame image that are captured at different points of time, according to an exemplary embodiment;

FIG. 9 illustrates a method for restoring a background region by using a graph obtained by mapping pixel information, according to an exemplary embodiment;

FIG. 10 is a flowchart illustrating a method for controlling an X-ray imaging apparatus in a case where a frame image includes a region of interest and a background region, according to an exemplary embodiment;

FIG. 11 is a block diagram of an X-ray imaging apparatus, which generates a frame image, the entire region of which corresponds to a region of interest, according to an exemplary embodiment;

FIG. 12 illustrates a method of summing, pixel-by-pixel, pixel information of frame images, according to an exemplary embodiment; and

FIG. 13 is a flowchart illustrating a method for controlling an X-ray imaging apparatus in a case where the entire region of a frame image generated by the X-ray imaging apparatus corresponds to a region of interest, according to an exemplary embodiment.

### DETAILED DESCRIPTION

Certain exemplary embodiments are described in greater detail below with reference to the accompanying drawings, wherein like reference numerals refer to like elements throughout. In this regard, the exemplary embodiments may have different forms and should not be construed as being limited to the descriptions set forth herein. Accordingly, the exemplary embodiments are merely described below, by referring to the drawings, to explain aspects of the present description. Sizes of elements in the drawings may be exaggerated for convenience of explanation. In other words, since sizes and thicknesses of components in the drawings are arbitrarily illustrated for convenience of explanation, the following exemplary embodiments are not limited thereto.

X-ray imaging apparatuses may have different structures or scanning schemes, depending on parts to be scanned, types of X-ray images, or scanning purposes. Specifically, the X-ray imaging apparatus in accordance with an exemplary embodiment may include any one of, for example, an X-ray imaging apparatus for scanning breast, arms, legs, etc., an X-ray imaging apparatus using mammography, which is a breast scanning technology, an X-ray imaging apparatus using fluoroscopy, an X-ray imaging apparatus using angiography, an X-ray imaging apparatus using cardiography, an X-ray imaging apparatus using tomography, and a combination thereof. For convenience of explanation, the X-ray imaging apparatus using fluoroscopy will be described as an example of the X-ray imaging apparatus in an exemplary embodiment. The X-ray imaging apparatus using fluoroscopy may obtain real-time videos, i.e., frame images, of an object by scanning the object with X-ray irradiation in various imaging modes. However, it should be noted that the X-ray imaging apparatus according to an exemplary embodiment may include any other types of X-ray imaging apparatuses.

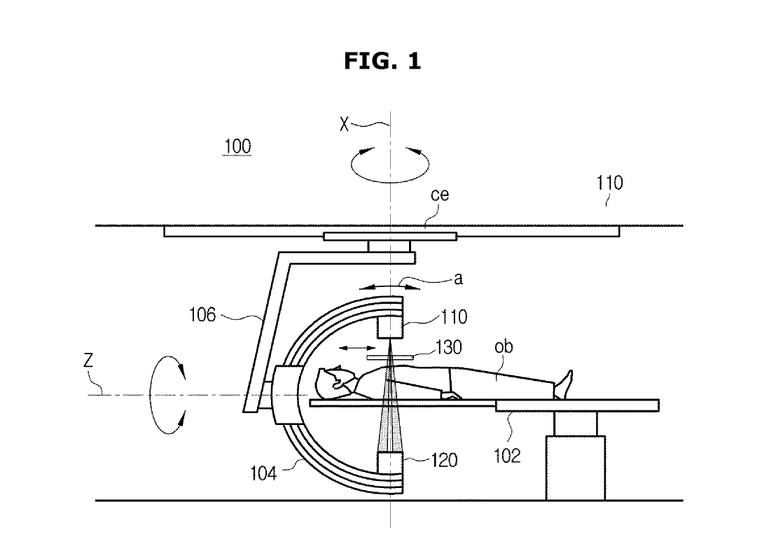

FIG. 1 illustrates an exterior appearance of an X-ray imaging apparatus according to an exemplary embodiment.

An X-ray imaging apparatus 100 may include an X-ray generator 110, and X-ray detector 120 arranged to face the X-ray generator 110. The X-ray generator 110 may generate X-rays and irradiate the X-rays to an object ob to obtain frame images of the object ob. More specifically, the X-ray generator 1110 may receive power from a power supply (not shown) to generate X-rays, wherein energy of the X-rays may be controlled by at least one of a tube voltage and a filter, and intensity or dose of X-rays may be controlled by tube current and X-ray exposure time.

The object ob as herein used may be, for example but not limited to, a living body of a human or an animal, or any object whose internal structure may be imaged by the X-ray imaging apparatus 100.

The X-ray detector 150 may detect X-rays that penetrate the object ob. The X-ray detector 150 may convert the detected X-rays to electrical signals, to X-ray data, and then to image signals, thereby generating frame images, which are X-ray images of the object ob.

Since the X-ray imaging apparatus 100 may scan the object ob to obtain real-time videos, i.e., frame images, of the object ob, reducing a dose of X-rays to be irradiated to the object ob is desirable to secure safety.

The X-ray imaging apparatus 100 may further include a filter 130. The X-ray imaging apparatus 100 may irradiate a higher dose of X-rays to a region of interest (ROI) among the entire region of a frame image while irradiating a lower dose of X-rays to a background region, through the filter 130, thereby reducing the total dose of radiation. In other words, the X-ray imaging apparatus 100 may reduce the total dose of radiation by irradiating a low dose of X-rays to a region outside the ROI, i.e., to the background region, through the filter 130.

The location of the ROI may change in time. For example, in a case where a doctor needs to intensively examine a part where a surgical operation tool, such as a catheter or a guidewire is located, the part where the tool is located may correspond to the ROI. As the doctor manipulates the tool, the location of the ROI may change. Accordingly, the X-ray imaging apparatus 100 may irradiate a high dose of X-rays only to the ROI by controlling movement of the filter 130 with a tracking algorithm. The tracking algorithm may be any algorithm for tracking movements of a surgical operation tool of interest or target part, which may be well-known to an ordinary skilled person in the art.

The X-ray imaging apparatus 100 may further include a table 102 to accommodate the object ob. Accordingly, the object ob lying on the table 102 may be located between the X-ray generator 110 and the X-ray detector 120 while X-rays are being irradiated by the X-ray generator 110.

As shown in FIG. 1, the X-ray generator 100 and the X-ray detector 120 may be located on opposite ends of a C-arm 104. The C-arm 104 may be installed to be able to rotate about a horizontal axis ‘Z’ as shown in FIG. 1. The C-arm 104 may also be rotated in a direction ‘a’ of FIG. 1, or in a semicircular manner.

Also, the C-arm 104 may be mounted on a supporter 106 installed on a ceiling ce, and the supporter 106 may be rotated about a vertical axis ‘X’. Accordingly, the X-ray imaging apparatus 100 may generate frame images of various ROIs of the object ob from different directions through rotation of the C-arm 104 and the supporter 106.

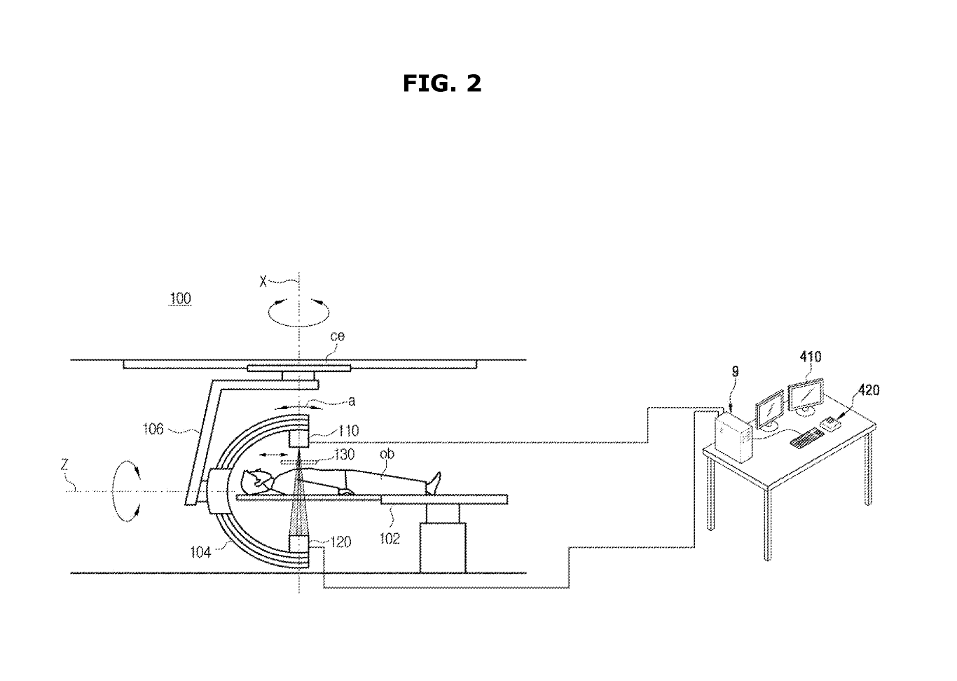

FIG. 2 illustrates an exterior appearance of an X-ray imaging apparatus including a console device, a display device, and an input device, according to an exemplary embodiment.

The X-ray generator 110, the X-ray detector 120, the C-arm 104, the supporter 106, the table 102, and the object ob of FIG. 2 are substantially the same as those described above, and thus the repetitive description will be omitted.

The X-ray imaging apparatus 100 may include a display 410. The display 410 may be implemented with liquid crystal displays (LCDs), light emitting diodes (LEDs), plasma display panels (PDPs), organic light emitting diodes (OLEDs), cathode ray tubes (CRTs), or the like. In a case where the display 410 is implemented in a type of a touch screen, the user may input various control instructions by touching the display 410.

The X-ray imaging apparatus 100 may also include an input device 420. The user may input various control instructions by manipulating the input device 420. The input device 420 may be implemented in various forms. For example, the input device 420 may be implemented with various buttons, a mouse, a keyboard, a trackball, a track pad, a stick, a handle, etc., but not limited thereto.

Referring to FIG. 2, the X-ray imaging apparatus 100 may include a console device 9. The console device 9 may be connected to at least one of the X-ray generator 110 and the X-ray detector 120 wired or wirelessly, for sending and/or receiving various control instructions and/or data to and/or from at least one of the X-ray generator 110, the X-ray detector 120, and the filter 130.

For example, the console device 9 may transmit various control instructions, which are input from the user through the display 410 implemented in a type of a touch screen or the input device 420, to at least one of the X-ray generator 110, the X-ray detector 120, and the filter 130. The X-ray generator 110, included in the X-ray imaging apparatus 100, will be described below.

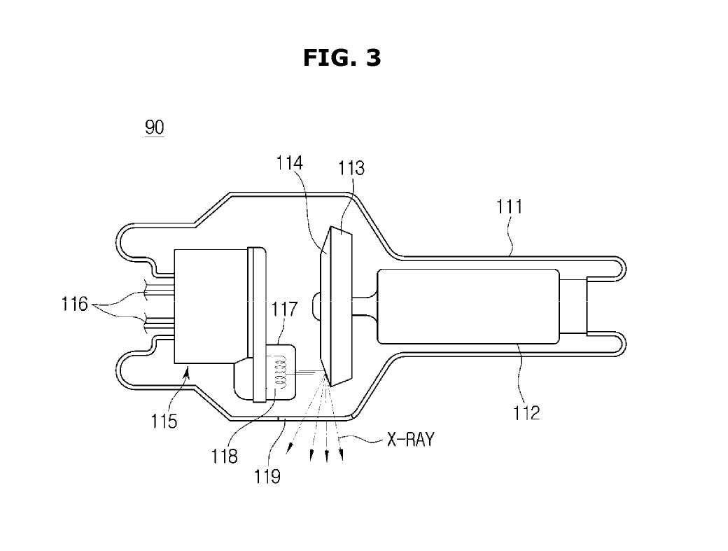

FIG. 3 illustrates a cross-section of an X-ray generator, according to an exemplary embodiment.

The X-ray generator 110 may include an X-ray tube 90 that produces X-rays, and the X-ray tube 90 is also called an X-ray tube head or X-ray tube assembly. The X-ray tube 90 may be implemented as a bipolar vacuum tube 111 that includes positive and negative electrodes 113 and 115, and the body of the vacuum tube 111 may be a glass tube including, e.g., hard silicon glass.

The negative electrode 115 may include a filament 118 and a focusing electrode 117 that focuses electrons, the focusing electrode 117 being also called a focusing cup. The focusing electrode 117 may form the inside of the glass tube 111 in a high vacuum state of about 10 mmHg, and produce thermoelectrons by heating the filament 118 of the negative electrode 115 at high temperatures. As an example of the filament 118, a tungsten filament may be used, which may be heated by applying a current to an electric wire 116 coupled with the filament 118.

The positive electrode 113 may include copper, and a target material 114 is applied or disposed on a side surface of the positive electrode 113 that faces the negative electrode 115, the target material 114 including a high resistive material, such as Cr, Fe, Co, Ni, W, Mo, or the like. The higher the melting point of the target material 114 is, the smaller a focal spot size may be.

When a high voltage is applied across the negative and positive electrodes 115 and 113, thermoelectrons are accelerated and collide with the target material 114 on the positive electrode 113, thus producing an X-ray. The X-ray may be irradiated through a window 119 that may be provided in a thin film of Beryllium. A filter (not shown) may be placed on the front or rear side of the window 119, to filter X-rays in a particular energy band.

The target material 114 may be rotated by a rotor 112. When the target material 112 is rotated, heat accumulation rate may increase more than ten times per unit area as compared with an occasion where the target material 114 is stationary, and the focal spot size may decrease.

A voltage applied across the negative and positive electrodes 115 and 113 of the X-ray tube 110 is called a tube voltage whose magnitude may be represented by a crest value kvp. As the tube voltage increases, the speed of thermoelectrons increases, and as a result, energy of X-rays (or energy of photon radiation) from collision of thermoelectrons with the target material 114 increases. Current flowing through the X-ray tube 110 is called a tube current, which may be represented by an average value mA. As the tube current increases, a dose of X-rays (or the number of photons of X-rays) also increases.

Accordingly, since the energy of X-rays may be controlled by the tube voltage while the intensity or dose of X-rays may be controlled by the X-ray exposure time, the energy and intensity of X-rays to be irradiated may be controlled based on types or characteristics of the object.

If X-rays to be irradiated have a certain energy band, the energy band may be defined by upper and lower limits. The upper limit of the energy band, i.e., a maximum energy of X-rays to be irradiated may be controlled by the level of the tube voltage, and the lower limit of the energy band, i.e., a minimum energy of X-rays to be irradiated may be controlled by a filter. Filtering X-rays of a low energy band by using the filter may increase the average energy of X-rays to be irradiated.

Although not shown, the X-ray generator 110 may further include a collimator placed on the front side of the window 110. The collimator may adjust an irradiation range of X-rays irradiated from the X-ray tube 110, and reduce X-ray scattering. Components of the X-ray imaging apparatus will now be described in more detail.

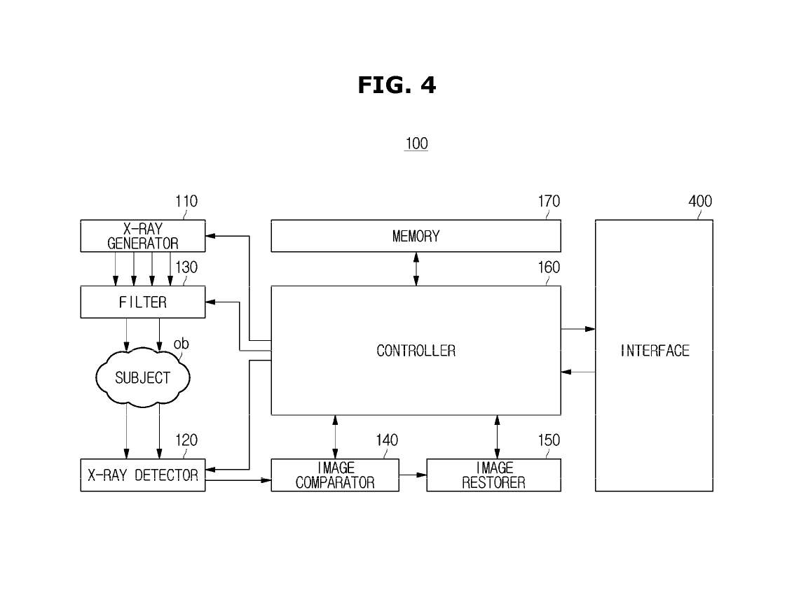

FIG. 4 is a block diagram of an X-ray imaging apparatus that provides different brightness levels for an ROI and a background region, according to an exemplary embodiment.

As shown in FIG. 4, the X-ray imaging apparatus 100 may include an X-ray generator 110, an X-ray detector 120, a filter 130, an image comparator 140, an image restorer (or image processor) 150, a controller 160, a memory 170, and an interface 400. The image comparator 140, the image restorer 150, the controller 160, and the interface 400 may be integrated on a system on chip (SOC) included in the X-ray imaging apparatus 100.

The X-ray generator 110, the X-ray detector 120, and the filter 130 are substantially the same as those described above, and thus the repetitive description will be omitted.

The image comparator 140 may compare frame images produced by the X-ray detector 120. Frame images generated by scanning an object may be continuously obtained over time during which the X-ray imaging apparatus 100 is activated. In the following description, a first frame image refers to a frame image obtained by scanning the object at a particular point of time t (t≥0), and a second frame image refers to a frame image obtained by scanning the object before the particular point of time t. For example, if the particular point of time is a current point of time, the first frame image may correspond to a current frame image, and the second frame image may correspond to a previous frame image.

The image comparator 140 may compare frame images produced by the X-ray detector 120 at different time points with each other. In this regard, it is possible to compare the frame images in all of the pixels contained in the frame images. However, since the X-ray generator 110 irradiates a high dose of X-rays to an ROI, the pixels contained in the ROI may have more accurate pixel information than the pixels in the background region. Accordingly, the image comparator 140 may use pixels of an ROI in the previous frame image to be compared with pixels of the background region in the current frame image.

The pixel information as herein used refers to information related to characteristics of pixels contained in a frame image. In an exemplary embodiment, the pixel information may include information about a brightness level, i.e., an intensity level of the pixel, and information about a noise level of the pixel. The pixel information is not limited thereto, but may include any types of various pieces of information obtainable from the frame image.

For example, the image comparator 140 may compare pixel information of the background region in the first frame image and pixel information of the ROI in the second frame image. In addition, the image comparator 140 may compare pixel information of the background region in the first frame image and pixel information of the ROI in the second frame image stored in e.g., a memory located within or outside of the X-ray imaging apparatus 100, or a web server.

Although in a case where the entire region of the frame image is fixed, the location of the ROI may be changed in time. In response, the location of the background region that corresponds to a region outside of the ROI may also be changed. In other words, a region corresponding to the background region in the first frame image may be a region corresponding to the ROI in the second frame image. Accordingly, with respect to pixels corresponding to the background region in the first frame image, the pixel information of the ROI of the second frame image captured with a high dose of X-rays may include more accurate and detailed information than pixel information for the background region of the first frame image captured with a low dose of X-rays. Therefore, by using the pixel information of the ROI irradiated with a high dose of X-rays in the second frame image captured before the particular point of time, more accurate restoration of the background region of the first frame image may be possible.

Restoration as herein used refers to a process to enhance the quality of an image. For example, restoration of the background region may refer to a process of compensating for a difference in brightness between the background region and the ROI of the image, and enhancing image quality of the background region using a noise cancellation scheme. As discussed above, the X-ray imaging apparatus may irradiate a high dose of X-rays only to the ROI while irradiating a low dose of X-rays to the background region by using the filter. This causes the ROI in the frame image to be brighter while rendering the background region dark and noisy, thereby increasing difficulties in identifying the object from the X-ray image before restoration. Accordingly, the X-ray imaging apparatus needs a process of restoration of the background region of an X-ray image.

The image comparator 140 may perform pixel by pixel mapping between the first frame image and some of a plurality of second frame images captured before the particular point of time. In other words, the image comparator 140 may perform pixel by pixel mapping between the first frame image and only on one or more of second frame images captured before the particular point of time.

Alternatively, the image comparator 140 may perform pixel by pixel mapping between the first frame image and all of the plurality of second frame images captured before the particular point of time. Still alternatively, the image comparator 140 may determine any one of the second frame images to be mapped with the first frame image, based on a user selection or a predetermined criterion, and then perform pixel by pixel mapping on the determined second frame image.

In an exemplary embodiment, with respect to a particular point of time t at which the first frame image is captured, the image comparator 140 may map, pixel by pixel, the pixel information of the ROI in the second frame image captured before the particular point of time t to the pixel information of the background region of the first frame image. The image comparator 140 may set coordinates of each mapping point between the first and second frame images and generate a graph based on the respective mapping points. In other words, the image comparator 140 may produce a graph, from which characteristics of pixels contained in the background region of the first frame image and the ROI of the second frame image may be determined.

In an exemplary embodiment, an X-axis of the graph may correspond to a brightness level of the background region of the first frame image and a Y-axis may correspond to a brightness level of the ROI of the second frame image. In other words, an X coordinate value of a point on the graph is determined based on the brightness level of the mapping pixel of the first frame image captured at a point of time t, and a Y coordinate value may be determined based on the brightness level of the mapping pixel of the second frame image captured at a point of time before time t.

If the object does not move and the entire region of the frame image is fixed, pixels on the same coordinates in a plurality of frame images captured at different points of time may have pixel information about the same region. Accordingly, the image comparator 140 may match pixels on the same coordinates in the plurality of frame images, to be mapped into a single point in the graph. In this regard, the X-ray imaging apparatus may generate the graph by using pixel information of a pixel mapped into each point to set XY coordinates for the point.

In an exemplary embodiment, to map pixels on the same coordinates in the first and second frame images into a point on a graph, the image comparator 140 may set an X-coordinate value of the point based on the brightness level of the pixel of the first frame image mapped into the point while setting a Y-coordinate value of the point based on the brightness level of the pixel of the second frame image mapped into the point, thereby determining the X and Y coordinates of the point on the graph. In this way, the image comparator 140 may produce a graph including points to which pixels included in the background region in the first frame image and the ROI in the second frame image are mapped.

However, when the object moves or a part for treatment is changed, the regions in the plurality of frame images captured at different points of time may be different. That is, pixels on the same coordinates in the plurality of frame images may have pixel information corresponding to different regions. The image comparator 140 may coordinate coordinates between pixels in the plurality of frame images through an image coordination process among the plurality of frame images. In the following description, the image coordination process refers to a process of coordinating coordinates among the plurality of frame images into one coordinate. For example, the image coordinating process among frame images may be performed, but not limited to, in a well-known method, such as elastic registration, non-rigid registration, etc.

The image comparator 140 may match pixels located on the same coordinates in the plurality of frame images, whose coordinates are coordinated through the image coordination process, to be mapped into a single point. In this regard, the image comparator 140 may generate the graph by setting coordinates of a point included in the graph using pixel information of the pixels mapped to the point. In an exemplary embodiment, the image comparator 140 may set an X coordinate value based on the brightness level of the pixel of the first frame image, which is mapped to the point, and set a Y coordinate value based on the brightness value of the pixel of the second frame image, which is mapped to the point. In this way, the image comparator 140 may generate the graph, in which the pixels belonging to the ROI of the first frame image and the background region of the second frame image are mapped.

The image comparator 140 may map pixel information of the background region of a frame image produced over time and pixel information of the ROI of a subsequent frame image, to a single point, set coordinates for the point, and represent the point in the graph, thereby updating the graph. Alternatively, the image comparator 140 may update the graph with respect to a frame image closer to the current frame image in time, by applying weights to the frame image closer to the current frame image in time. As such, there may be various methods for setting coordinates, and there is no limitation to the methods for setting coordinates.

The image comparator 140 may use the graph to determine a difference between the brightness levels of mapping pixels at a particular time and before the particular time, a difference between the noise levels of the mapping pixels, etc. Also, the image comparator 140 may determine to what extent the brightness level needs to be compensated, to what extent the noise level needs to be set, etc., in restoring the background region of the first frame image.

The image restorer 150 may restore the background region of the first frame image by using the determined noise levels and brightness levels. In other words, the image restorer 150 may restore the background region more precisely by using pixel information of pixels contained in a previously captured frame image.

The image restorer 150 may restore the background region in various methods based on information obtainable from the graph. In an exemplary embodiment, the X axis and Y axis of the graph correspond to a brightness level of the background region of the first frame image and a brightness level of the ROI of the second frame image, respectively. Thus, respective points represent correspondence between the first and second frame images. The image restorer 150 may thus restore respective pixels of the background region of the first frame image through the mapping between the first and second frame images. Specifically, the image restorer 150 may restore each pixel of the first frame image by determining an extent of compensation for a brightness level, and an extent to which the noise level for the pixel is to be set, based on the mapping between the first and second frame images.

In another example, the image restorer 150 may restore the background region of the first frame image by representing a distribution pattern of points on the graph in a finite number of parameters to set constraint values of the brightness level and noise level of pixels of the first frame image. In addition, the image restorer 150 may restore the background region of the first frame image in various methods based on information obtainable from the graph.

The controller 160 may send control signals to respective components to perform respective functions of the X-ray imaging apparatus 100, upon reception of control instructions from the user. The controller 160 may control overall operations and signal flows of internal components of the X-ray imaging apparatus 100, and perform a function to process data.

The controller 160 may also control power supplied from a power supply to be distributed to the internal components of the X-ray imaging apparatus 100. Moreover, the controller 160 may control a dose, intensity, and direction of X-rays to be irradiated to the object. For example, the controller 160 may be implemented as a processor such as a central processor unit (CPU), a micro controller unit (MCU), or a micro processor unit (MPU). Although the image comparator 140 and the image restorer 150 are illustrated as separate elements from the controller 160 in FIG. 4, the exemplary embodiments are not limited thereto. In an exemplary embodiment, some or all of functions of the image comparator 140 and the image restorer 150 may be performed by the controller 160.

The X-ray imaging apparatus 100 may include the interface 400. The interface 400 may display frame images on the display 410. The interface 400 may receive at least one of various control instructions from the user and/or various data through a input device. The input device 420 may be implemented with various buttons, a mouse, a keyboard, a trackball, a track pad, a stick, a handle, etc., but is not limited thereto. If the display 410 is implemented in the type of a touch screen, the display 410 may also perform functionality of the input device 420. In this case, the interface 400 may include a graphic user interface (GUI) to receive a user's command.

That is, the interface 400 may provide frame images for the user or receive various control instructions from the user, in conjunction with all the devices that may provide frame images for the user or receive various control instructions from the user.

The memory 170 may store frame images captured in real time. The memory 170 may be implemented by a random-access memory (RAM), a read-only memory (ROM), a flash memory, and a memory card in a card type, such as a secure digital (SD) card, a solid state drive (SSD) card, etc.

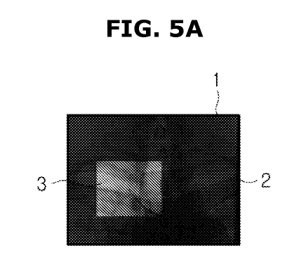

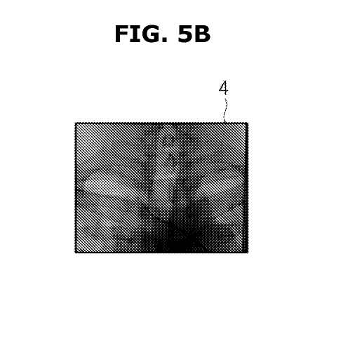

FIGS. 5A and 5B illustrate frame images before and after restoration of a background region, according to an exemplary embodiment.

FIG. 5A shows a frame image 1 before restoration of a background region 2, and FIG. 5B shows a frame image 4 after restoration of the background region 2.

Referring to FIG. 5A, the X-ray imaging apparatus may open the filter and irradiate a high dose of X-rays only for an ROI 3. In contrast, the X-ray imaging apparatus may irradiate a low dose of X-rays for the background region 2 by using the filter. As a result, as shown in FIG. 5A, the ROI 3 in the frame image 1 is bright while the background region 2 is dark and noisy, which causes difficulties in identifying the object before restoration.

Referring to FIG. 5B, the X-ray imaging apparatus may provide the frame image 4 obtained by restoring the background region 2. In restoring the background region 2, to what extent the brightness level of the background region 2 needs to be compensated and to what extent the noise needs to be eliminated from the background region 2 may be determined.

In this regard, the X-ray imaging apparatus may use pixel information of the captured frame images to more precisely determine the extent of the compensation of the brightness level of the background region 2 and/or the extent of noise elimination in the background region 2. As a result, the X-ray imaging apparatus may more precisely restore the background region 2.

For example, the X-ray imaging apparatus may include an image acquirer configured to acquire a plurality of frame images of an object at different times, and an image processor configured to map a pixel in a background region of a current frame image to a pixel in a region of interest of at least one previous frame image, and compensate the background region of the current frame image based on pixel information of a mapping pixel in the region of interest of the at least one previous frame image.

The image processor may compensate the background region of the current frame image based on at least one of a difference between brightness levels between noise levels of mapping pixels between the current frame image and the at least one previous frame image and a difference between noise levels of mapping pixels between the current frame image and the at least one previous frame image. The image processor may map pixels at the same coordinates between the background region of the current frame image and the region of interest of the at least one previous frame image. When the current frame image and the at least one previous frame image correspond to different regions of the object, the image processor may perform an image coordination process to adjust coordinates of pixels of the current frame image and the at least one previous frame image, and the image processor may map pixels at the same coordinates between the current frame image and the at least one previous frame image on which the image coordination process is performed.

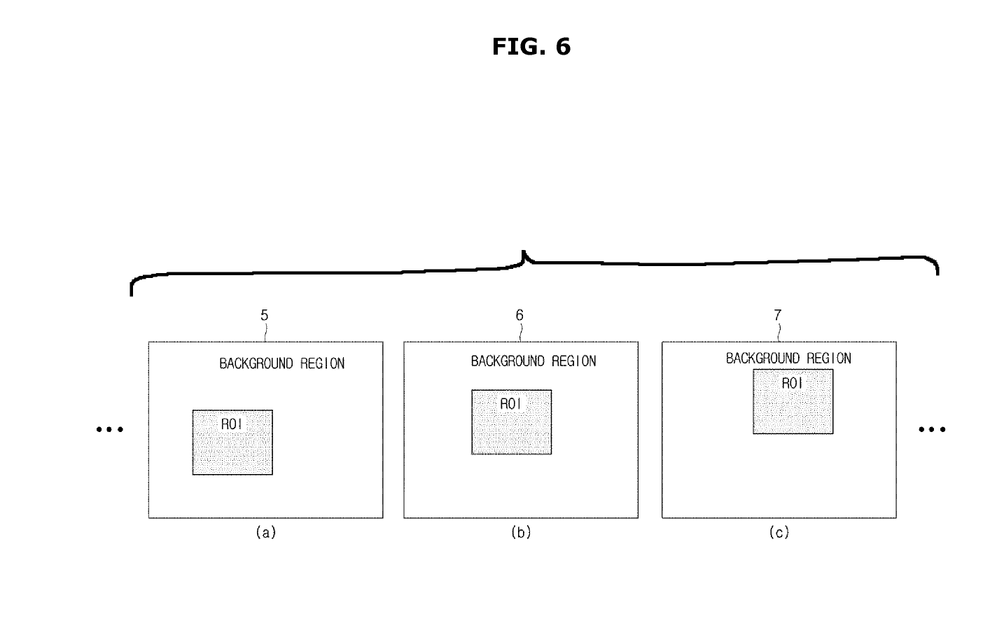

FIG. 6 illustrates an ROI moving in frame images over time, according to an exemplary embodiment.

In FIG. 6, (a) shows a frame image 5 at time (t-2), (b) shows a frame image 6 at time (t-1), and (c) shows a frame image 7 at time t.

Assuming that the entire region of each of the frame image 5, 6, 7 is fixed, an ROI may be changed within the frame images 5, 6, and 7, as shown in FIG. 6. In response, the background region that corresponds to a region outside of the ROI, may also be changed.

Accordingly, there may be a region that corresponds to an ROI at least once in the previous frame image and now corresponds to the background region in the current frame image. For example, an ROI of the frame image 5 at time (t-2) may correspond to a background region in the frame image 6 at (t-1). In another example, an ROI of the frame image 6 at (t-1) may correspond to a background region in the frame image 7 at t. Accordingly, the X-ray imaging apparatus may more precisely determine a background region in the current frame image by using pixel information of the ROI in a previously captured frame image. The X-ray imaging apparatus may thus more precisely restore the background region of the current frame image.

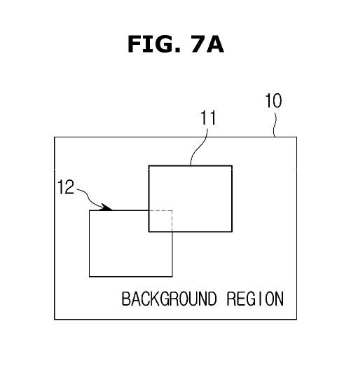

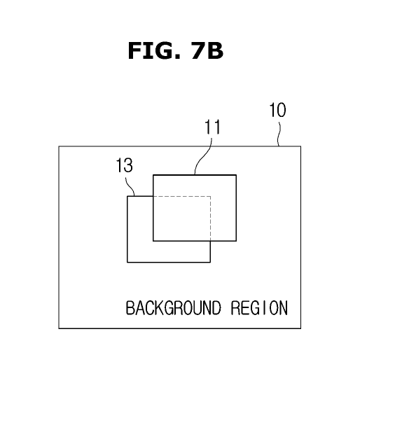

FIGS. 7A and 7B illustrate relationships between a first frame image obtained by scanning an object at a particular point of time and a second frame image obtained by scanning the object before the particular point of time, according to an exemplary embodiment.

For a frame image 10, the entire region of which is fixed, FIG. 7A shows the frame image 10 containing an ROI 11 at time t and an ROI 12 at time (t-2). FIG. 7B shows the frame image 10 containing the ROI 11 at time t and an ROI 13 at time (t-1).

Referring to FIG. 7A, the X-ray imaging apparatus may restore a background region of the frame image 10 by using pixel information of an area overlapping between a region corresponding to the ROI 12 at time (t-2) and a region that corresponds to the background region at time t. Similarly, referring to FIG. 7B, the X-ray imaging apparatus may restore a background region of the frame image 10 by using pixel information of an area overlapping between a region corresponding to the ROI 13 at time (t-1) and the region that corresponds to the background region at time t.

That is, at t, the X-ray imaging apparatus may irradiate a high dose of X-rays to the ROI, and thus obtain accurate image of the ROI. In contrast, the background region is irradiated by a low dose of X-rays through the filter, and thus appear to be dark, which causes difficulties in obtaining pixel information of the background region. Accordingly, the X-ray imaging apparatus may produce a graph using the pixel information of pixels that belong to the ROI in a frame image captured before time t, and determine imaging parameters to be used to restore the background region of the frame image at time t, based on statistical distribution obtainable from the graph.

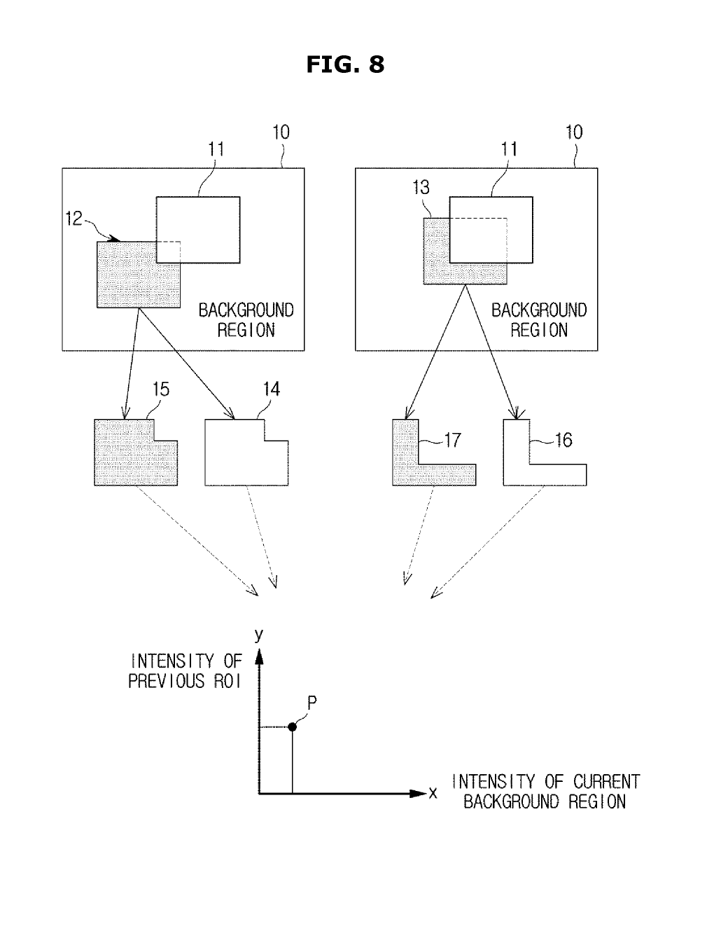

FIG. 8 illustrates an example of producing a graph by mapping pixel information between a first frame image obtained by scanning an object at a particular point of time and a second frame image obtained by scanning the object before the particular point of time, according to an exemplary embodiment.

As described above, for a frame image 10, the entire region of which is fixed, the frame image 10 on the left of FIG. 8 contains an ROI 11 at time t and an ROI 12 at time (t-2). The frame image 10 on the right of FIG. 8 contains the ROI 11 at time t and an ROI at time (t-1).

Referring to FIG. 8, for pixels 15 and 14 that belong to the ROI 12 at time (t-2) and to the background region at time t, respectively, the X-ray imaging apparatus may match pixels 15 having pixel information at time (t-2) and pixels 14 having pixel information at time t, pixel by pixel, on the same coordinates, and map the matched pixels into respective points on X and Y axes. In this regard, when the object does not move and the entire region is fixed, the X-ray imaging apparatus may match the pixels 14 and 15, pixel by pixel, on the same coordinates, and map the matched pixels into respective points on X and Y axes. On the other hand, when the entire region is not fixed, the X-ray imaging apparatus may coordinate coordinates of pixels 15 having pixel information at time (t-2) and pixels 14 having pixel information at time t through an image coordination process, and map pixels on the same coordinates after the image coordination process has been performed into respective points on X and Y axes.

The X-ray imaging apparatus may use the pixel information of pixels mapped into each point to set XY coordinates of the point, thereby representing the respective points on the graph. For example, the X-ray imaging apparatus may map pixels 17 and 16 in the region that corresponds to the ROI 13 at time (t-1) and corresponds to the background region at time t into respective points, including a point P, to be represented in the graph.

As such, the X-ray imaging apparatus may produce a graph as shown in the lower part of FIG. 8. The X-axis of the graph represents a brightness level of the background region at time t, i.e., intensity of the current background region, and the Y-axis represents a brightness level of the ROI before time t, i.e., intensity of the previous ROI. By using the graph, the X-ray imaging apparatus may determine a correspondence relationship between brightness levels of the current background region and the previous ROI.

In an exemplary embodiment, the X-ray imaging apparatus may determine noise distribution from the statistical distribution of the points on the graph, and in modeling the noise distribution and restoring the background region, determine limits of the noise level of the restored background region.

In another exemplary embodiment, the X-ray imaging apparatus may directly set a brightness level and a noise level per pixel by using mapping results of information regarding correspondence between brightness levels of the current background region and the previous ROI, thereby restoring respective pixels in the background region. The X-ray imaging apparatus may restore the current background region in various methods by using pixel information of pixels that correspond to the previous ROI, and there is no limitation to the methods of restoring the current background region.

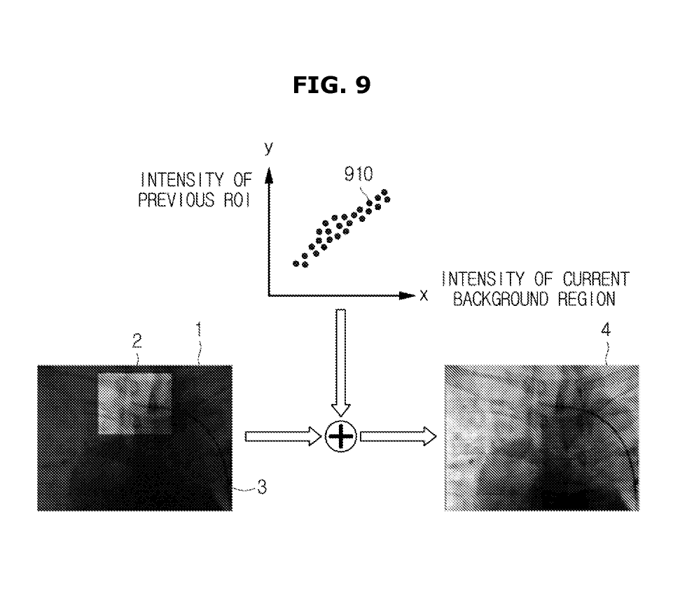

FIG. 9 illustrates a method for restoring a background region with a graph produced by mapping pixel information, according to an exemplary embodiment. As shown in FIG. 9, the X-ray imaging apparatus may separately capture an ROI 2 and a background region 3. In restoring the background region 3 in a frame image 1, the X-ray imaging apparatus may restore the background region 3 more precisely by using information obtained from a graph 910. Specifically, the X-ray imaging apparatus may determine tendency of noise distribution and difference in brightness levels between the current background region and the previous ROI from the graph 910, and use the same to restore the background region 3. There is no limitation to the background region restoration method as long as pixel information for the previous ROI may be used in restoring the background region 3. As a result, the X-ray imaging apparatus may generate a frame image 4 obtained by restoring the background region 3.

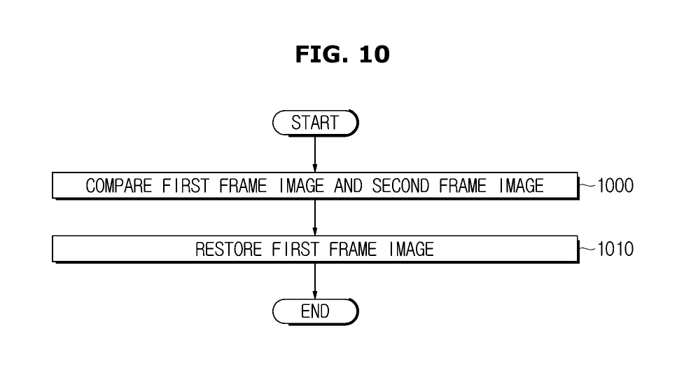

FIG. 10 is a flowchart illustrating a method for controlling an X-ray imaging apparatus in a case where a frame image includes an ROI and a background region, according to an exemplary embodiment.

The X-ray imaging apparatus may generate a frame image by irradiating X-rays to scan an internal part of an object. The X-ray imaging apparatus may obtain the frame image with an ROI and a background region by controlling the filter. Specifically, the X-ray imaging apparatus may reduce the total dose of radiation by setting a high dose of X-rays only for a desired part of the object. Accordingly, the region scanned with a high dose of X-rays without the filter, i.e., the ROI, has a higher resolution and has less noise. In contrast, the background region that corresponds to a region outside of the ROI has a lower resolution and has more noise.

The X-ray imaging apparatus may compare the first frame image obtained by scanning the object at a particular point of time and one or more second frame images obtained by scanning the object before the particular point of time, in operation 1000. The first and second frame images are each divided into an ROI and a background region, the ROIs or background regions in the frame images being or not being identical to each other.

The X-ray imaging apparatus may obtain pixel information of the ROI of the second frame image. The X-ray imaging apparatus may determine characteristics of pixels belonging to the ROI of the second frame image from the pixel information of the ROI scanned by irradiating a high dose of radiation as compared to the other region in the second frame image.

Therefore, in a case where it is difficult to determine characteristics of pixels belonging to the background region of the first frame image, the X-ray imaging apparatus may use the pixel information of the ROI of the second frame image to accurately determine characteristics of the pixels of the background region of the first frame image.

The X-ray imaging apparatus may map the pixel information of the background region of the first frame image and the pixel information of the ROI of the second frame image into a single point for each pixel, set coordinates for the respective points, and represent the points on the graph. In an exemplary embodiment, the graph may be a two dimensional (2D) graph. The X-axis of the graph may represent a brightness level per pixel of the background region of the first frame image and the Y-axis may represent a brightness level per pixel of the ROI of the second frame image. The X-ray imaging apparatus may set coordinates for each pixel based on the brightness level of the pixel of the background region of the first frame image and the brightness level of the pixel of the ROI of the second frame image, to represent points of the coordinates in the graph.

Accordingly, the X-ray imaging apparatus may determine statistical distribution of the points represented in the graph. In an exemplary embodiment, the X-ray imaging apparatus may project the points onto the X-axis or Y-axis, and then calculate a variance related to the distribution of the points. The X-ray imaging apparatus may use the calculation result to determine tendency of distribution of a brightness level per pixel, tendency of distribution of a noise level per pixel, etc., in the background region of the first frame image. In addition, the X-ray imaging apparatus may use various information derived from the graph.

The X-ray imaging apparatus may use the information derived from the graph to determine brightness levels to be set for restoring the pixels belonging to the background region of the first frame image. The X-ray imaging apparatus may also use the information derived from the graph to determine noise levels to be set for restoring the pixels belonging to the background region of the first frame image. The X-ray imaging apparatus may thus more accurately restore the background region of the first frame image, in operation 1010. When the entire region of the frame image is changed due to a movement of the patient (or object), a change in a treatment part, etc., the X-ray imaging apparatus may perform a process of the image coordination between the first and second frame images, and then map the pixel information of each pixel of the coordinated image into a single point.

The X-ray imaging apparatus may display the restored first frame image on a display. The X-ray imaging apparatus may display the restored first frame image on the display 410 included in the X-ray imaging apparatus, as shown in FIG. 2. Alternatively, the X-ray imaging apparatus may be wired or wirelessly connected to a display located outside, for displaying the restored first frame image. With respect to a frame image whose entire region corresponds to an ROI, operation of the X-ray imaging apparatus will now be described.

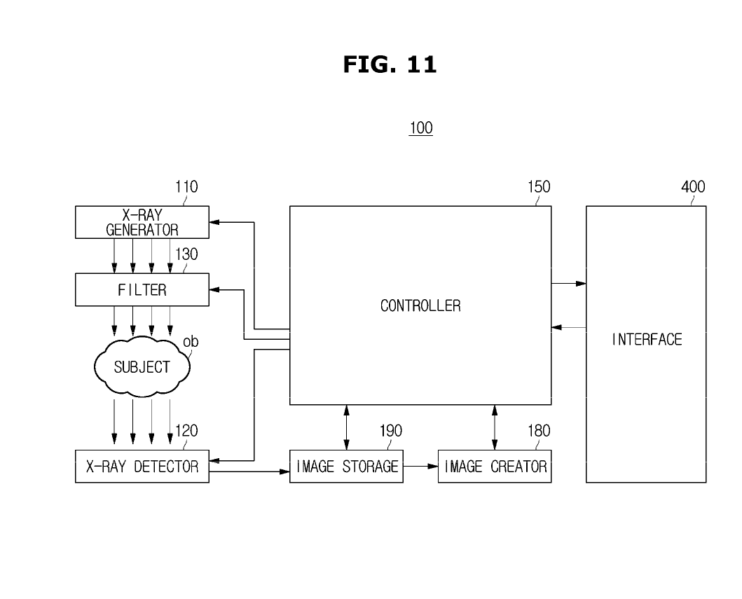

FIG. 11 is a block diagram of an X-ray imaging apparatus, which generates a frame image, the entire area of which corresponds to an ROI, according to an exemplary embodiment.

Referring to FIG. 11, the X-ray imaging apparatus 100 may include an X-ray generator 110, an X-ray detector 120, a filter 130, a controller 150, an image storage 190, an image generator 180, and an interface 400. The controller 150, the image storage 190, the image generator 180, and the interface 400 may be integrated in an SOC embedded in the X-ray imaging apparatus 100. The X-ray generator 110, the X-ray detector 120, and the filter 130 are substantially the same as those described above, and thus the repetitive description will be omitted.

The image storage 190 may store frame images continuously produced over time in a memory. The image storage 190 may store the frame images in a memory embedded in the X-ray imaging apparatus 100 for storing data, or in an external memory. The memory 170 may be implemented by an RAM, an ROM, a flash memory, and a memory card in a card type, such as a secure digital (SD) card, a solid state drive (SSD) card, etc.

The image generator 180 may generate a first frame image, which is brighter and less noisy, using pixel information obtained from the stored frame images. For example, when the entire region of the frame image corresponds to an ROI, the image generator 180 may obtain, pixel by pixel, a sum of pixel information of the second frame images captured before a particular point of time.

The pixel information as herein used refers to information about a plurality of pixels contained in the frame image. In an exemplary embodiment, the pixel information may include information about a brightness level, i.e., an intensity level of the pixel, and information about a noise level of the pixel. The pixel information is not limited thereto, but may include non-limited various pieces of information obtainable in the image.

In an exemplary embodiment, the image generator 180 may determine the brightness level of each pixel, by summing, pixel by pixel, brightness levels of second frame images captured before the particular point of time. In another exemplary embodiment, the image generator 180 may determine to the noise level of each pixel, by summing, pixel by pixel, noise levels of the second frame images captured before the particular point of time. The image generator 180 may thus more accurately restore the present frame image by using pixel information of the previous frame images obtained by scanning the same region.

In an exemplary embodiment, pixel information of one of the second frame images, which is adjacent to the first frame image in time, may be considered more useful than the other second frame images. Accordingly, the image generator 180 may apply a weight to the pixel information obtained from the closest second frame image. There may be various other ways for the image generator 180 to generate the first frame image by using the pixel information obtained from the second frame image.

On the other hand, the region of the frame image may not be fixed. For example, the region of the frame image may vary according to movement of the patient or shift in a treatment part. In this regard, the image generator 180 may first perform a process of image coordination between the second frame images, and then sum pixel information of the second frame images, pixel by pixel. Accordingly, when the region of the frame image is not fixed, the image generator 180 may use the pixel information of the second frame images obtained through the image coordination process in generating the first frame image.

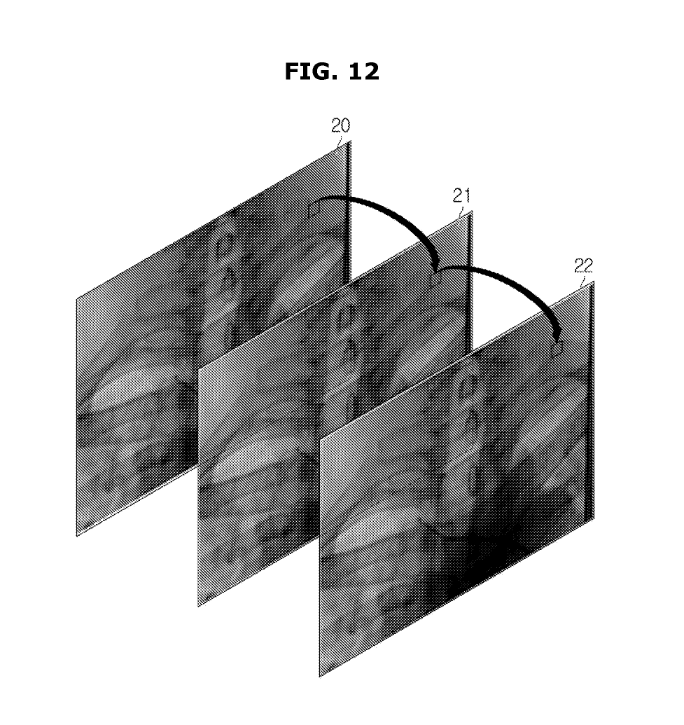

FIG. 12 illustrates a method of summing pixel-by-pixel pixel information of frame images, according to an exemplary embodiment.

Referring to FIG. 12, the X-ray imaging apparatus may continuously capture frame images. In an exemplary embodiment, a frame image 20 corresponds to a frame image captured at time (t-3), a frame image 21 corresponds to a frame image captured at time (t-2), and a frame image 22 corresponds to a frame image captured at time (t-1).

In an exemplary embodiment where the entire region of the frame image is fixed, the X-ray imaging apparatus may obtain pixel information from respective pixels of the frame images 20, 21, and 22 that are captured before time t. The X-ray imaging apparatus may sum the obtained pixel information, pixel by pixel. Accordingly, the X-ray imaging apparatus may obtain all the pixel information of the pixels contained in the plurality of frame images 20, 21, and 22.

The X-ray imaging apparatus may statistically determine the brightness level, i.e., intensity, and noise level of each pixel from the obtained pixel information. Accordingly, the X-ray imaging apparatus may generate more accurate frame images by additionally using the statistical information.

In another exemplary embodiment, the region of the frame image may vary according to movement of the patient or shift in a treatment part. In this regard, the image generator 180 may first perform the process of image coordination between frame images. Accordingly, the image generator 180 may obtain pixel information from the coordinated frame image, and perform an operation of summing pixel information for corresponding pixels.

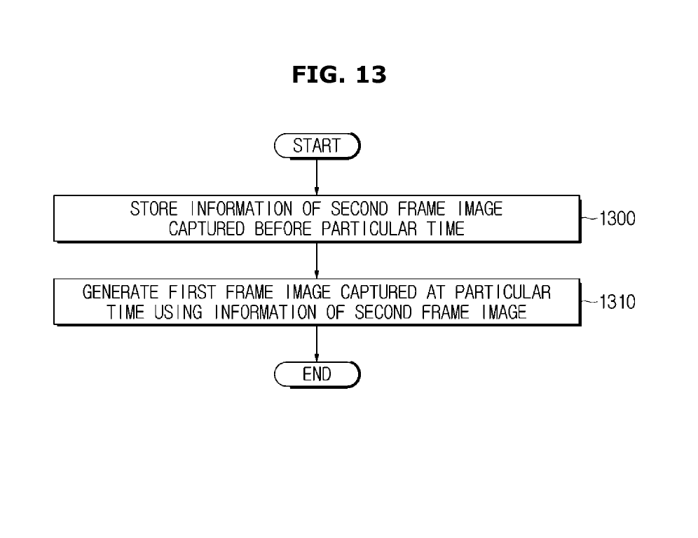

FIG. 13 is a flowchart illustrating a method for controlling an X-ray imaging apparatus in a case where the entire region of a frame image corresponds to an ROI, according to an exemplary embodiment.

The X-ray imaging apparatus may generate a frame image by irradiating X-rays to scan an internal part of an object. The X-ray imaging apparatus may obtain the frame image with an ROI and a background region by controlling the filter. Specifically, the X-ray imaging apparatus may reduce the total dose of radiation by setting a high dose of X-rays only for a desired part of the object. When the entire region of the frame image is an ROI, the X-ray imaging apparatus may set a higher dose of X-rays for the entire region of the frame image.

The X-ray imaging apparatus may store frame images obtained over time. For example, the X-ray imaging apparatus may store the frame images in a memory embedded in the X-ray imaging apparatus, or in an external memory. In another example, the X-ray imaging apparatus may be connected to a web storage or cloud server that has a storage function on the Web, and store the frame images in the web storage or cloud server.

The X-ray imaging apparatus may analyze the stored frame images, and obtain more accurate new frame images based on the analysis result. The second frame image as herein used refers to frame images obtained before a particular point of time and stored in the X-ray imaging apparatus, and the first frame image refers to an image to be obtained at the particular point of time.

The X-ray imaging apparatus may obtain information in generating the first frame image by applying an operation process for the obtained pixel information. In an exemplary embodiment, the X-ray imaging apparatus may determine the brightness level of each pixel, by summing brightness levels of second frame images captured before the particular point of time, pixel by pixel. In another exemplary embodiment, the X-ray imaging apparatus may determine the noise level of each pixel, by summing noise levels of the second frame images captured before the particular point of time, pixel by pixel. The X-ray imaging apparatus may thus more precisely restore the present frame image by using pixel information of the previous frame images obtained by scanning the same region.

Even when the entire region of the frame image is not fixed, the X-ray imaging apparatus may coordinate a plurality of frame images through an image coordination process as described above. Accordingly, the X-ray imaging apparatus may obtain pixel information of respective pixels, and use the pixel information to generate the first frame image.

The method according to the exemplary embodiments may be implemented in program instructions which are executable by various computing means (e.g., a computer) and recorded in computer-readable media. The computer-readable media may include program instructions, data files, data structures, etc., separately or in combination. The program instructions recorded on the computer-readable media may be designed and configured specially for the present disclosure, or may be well-known to people having ordinary skill in the art of computer software. Examples of the computer readable recording medium include read-only memory (ROM), random-access memory (RAM), Compact Disc (CD)-ROMs, magnetic tapes, floppy disks, optical data storage devices, etc. The computer readable recording medium can also be distributed over network coupled computer systems so that the computer readable code is stored and executed in a distributed fashion.

Examples of the program instructions include not only machine language codes but also high-level language codes which are executable by various computing means using an interpreter. The aforementioned hardware devices may be configured to operate as one or more software modules to carry out exemplary embodiments, and vice versa.

Although a few embodiments have been shown and described, it would be appreciated by those skilled in the art that changes may be made in these exemplary embodiments without departing from the principles and spirit of the disclosure, the scope of which is defined in the claims and their equivalents.

What is claimed is:

1. An X-ray imaging apparatus comprising:
an image comparator configured to compare a first frame image obtained by scanning an object at a particular point of time, and one or more second frame images obtained by scanning the object before the particular point of time; and
an image restorer configured to restore a background region of the first frame image based on a result of the comparison between the first frame image and the one or more second frame images.

2. The X-ray imaging apparatus of claim 1, wherein the image comparator is configured to map, pixel by pixel, pixel information of the background region of the first frame image and pixel information of a region of interest of the one or more second frame images.

3. The X-ray imaging apparatus of claim 2, wherein the image comparator is configured to generate a graph including one or more points, each of the one or more points being mapped, per pixel, between the pixel information of the background region of the first frame image and the pixel information of the region of interest of the one or more second frame images.

4. The X-ray imaging apparatus of claim 3, wherein the image comparator is configured to determine at least one of a brightness level per pixel and a noise level per pixel of the background region of the first frame image, based on the graph.

5. The X-ray imaging apparatus of claim 3, wherein the image comparator is configured to determine a constraint value for at least one of a brightness level per pixel and a noise level per pixel of the background region of the first frame image, based on the graph.

6. The X-ray imaging apparatus of claim 1, wherein the image restorer is configured to restore the background region of the first frame image by using brightness levels of the background region of the first frame image that are determined based on the result of the comparison between the first frame image and the one or more second frame images.

7. The X-ray imaging apparatus of claim 1, wherein the image restorer is configured to restore the background region of the first frame image by using noise levels of the background region of the first frame image that are determined based on the result of the comparison between the first frame image and the one or more second frame images.

8. The X-ray imaging apparatus of claim 1, wherein the image restorer is configured to restore the background region of the first frame image by using a constraint value for at least one of a brightness level and a noise level of the background region of the first frame image, the constraint value being determined based on the result of the comparison between the first frame image and the one or more second frame images.

9. The X-ray imaging apparatus of claim 1, further comprising:
a display configured to display the restored first frame image.

10. An X-ray imaging apparatus comprising:
an image storage configured to store pixel information of one or more second frame images obtained by scanning an object before a particular point of time; and
an image generator configured to generate a first frame image obtained by scanning the object at the particular point of time, by using the pixel information of the stored one or more second frame images.

11. The X-ray imaging apparatus of claim 10, wherein the image generator is configured to generate the first frame image by using a result of summing, pixel by pixel, pixel information of the stored one or more second frame images.

12. The X-ray imaging apparatus of claim 11, wherein the image generator is configured to calculate, pixel by pixel, a sum of respective brightness levels of pixels of the one or more second frame images, and generate the first frame image by using the sum of the respective brightness levels of the pixels of the one or more second frame images.

13. The X-ray imaging apparatus of claim 11, wherein the image generator is configured to calculate, pixel by pixel, a sum of respective noise levels of pixels of the one or more second frame images, and generate the first frame image by using the sum of the respective noise levels of the pixels of the one or more second frame image.

14. The X-ray imaging apparatus of claim 10, further comprising:
a display configured to display the generated first frame image.

15. A method for controlling an X-ray imaging apparatus, the method comprising:
comparing a first frame image obtained by scanning an object at a particular point of time, and one or more second frame images obtained by scanning the object before the particular point of time; and
restoring a background region of the first frame image based on a result of the comparing.

16. The method of claim 15, wherein the comparing comprises:
mapping, pixel by pixel, pixel information of a background region of the first frame image and pixel information of a region of interest of the one or more second frame images.

17. The method of claim 16, wherein the comparing comprises:
generating a graph including one or more points, each of the one or more points being mapped, per pixel, between the pixel information of the background region of the first frame image and the pixel information of the region of interest of the one or more second frame images.

18. The method of claim 17, wherein the comparing comprises:
determining at least one of a brightness level per pixel and a noise level per pixel of the background region of the first frame image, based on the graph.

19. The method of claim 17, wherein the comparing comprises:
determining a constraint value for at least one of a brightness level per pixel and a noise level per pixel of the background region of the first frame image, based on the graph.

20. The method of claim 15, wherein the restoring comprises:
restoring the background region of the first frame image by using brightness levels of the background region of the first frame image determined based on the result of the comparing.

21. The method of claim 15, wherein the restoring comprises:
restoring the background region of the first frame image by using noise levels of the background region of the first frame image determined based on the result of the comparing.

22. The method of claim 15, wherein the restoring comprises:
restoring the background region of the first frame image by using a constraint value for at least one of a brightness level and a noise level of the background region of the first frame image, the constraint value being determined based on the result of the comparing.

23. A method for controlling an X-ray imaging apparatus, the method comprising:
storing pixel information of one or more second frame images obtained by scanning an object before a particular point of time; and
generating a first frame image obtained by scanning the object at the particular point of time, by using the pixel information of the stored one or more second frame images.

24. The method of claim 23, wherein the generating comprises:
generating the first frame image by using a result of summing, pixel by pixel, pixel information of the stored one or more second frame images.

25. The method of claim 24, wherein the generating comprises:
calculating, pixel by pixel, a sum of respective brightness levels of pixels of the one or more second frame images, and generating the first frame image by using the calculated sum of the brightness levels of the pixels of the one or more second frame images.

26. The method of claim 24, wherein the generating comprises:
calculating, pixel by pixel, a sum of respective noise levels of pixels of the one or more second frame images, and generating the first frame image by using the calculated sum of the noise levels of the pixels of the one or more second frame images.

27. An X-ray imaging apparatus comprising:
an image acquirer configured to acquire a plurality of frame images of an object at different times; and
an image processor configured to map a pixel in a background region of a current frame image to a pixel in a region of interest of at least one previous frame image, and compensate the background region of the current frame image based on pixel information of a mapping pixel in the region of interest of the at least one previous frame image.

28. The X-ray imaging apparatus of claim 27, wherein the image processor is configured to compensate the background region of the current frame image based on at least one of a difference between brightness levels between noise levels of mapping pixels between the current frame image and the at least one previous frame image and a difference between noise levels of mapping pixels between the current frame image and the at least one previous frame image.

29. The X-ray imaging apparatus of claim 27, wherein the image processor is configured to map pixels at the same coordinates between the background region of the current frame image and the region of interest of the at least one previous frame image.

30. The X-ray imaging apparatus of claim 27, when the current frame image and the at least one previous frame image correspond to different regions of the object, the image processor is configured to perform an image coordination process to adjust coordinates of pixels of the current frame image and the at least one previous frame image, and the image processor is configured to map pixels at the same coordinates between the current frame image and the at least one previous frame image on which the image coordination process is performed.
